(19) United States
(12) Patent Application Publication
(10) Pub. No.: US 2012/0052064 A1
(43) Pub. Date: Mar. 1, 2012

(54) ANTI-HGF ANTIBODY COMBINATIONAL CANCER THERAPIES

(76) Inventors: Yuuki Ito, Kanagawa (JP); Satoru Nishizawa, Kanagawa (JP); Osamu Kitahara, Kanagawa (JP); Akira Hori, Kanagawa (JP)

(21) Appl. No.: 13/264,850

(22) PCT Filed: Apr. 16, 2010
(86) PCT No.: PCT/JP2010/057199

§ 371 Date: Nov. 15, 2011

Related U.S. Application Data
(60) Provisional application No. 61/202,892, filed on Apr. 17, 2009.

(51) Int. Cl. A61K 39/395; A61P 35/00

(52) U.S. Cl. 424/133.1; 424/145.1; 424/142.1

(57) ABSTRACT

The present invention relates to the treatment of cancer (tumor) by administering to a patient in need of such treatment a first agent that inhibits Hepatocyte Growth Factor (HGF) in combination with a second agent that inhibits a signaling pathway other than the one stimulated by HGF.

### FIELD OF THE INVENTION

The present invention relates generally to the treatment of cancer (tumor), and more particularly, for example, to treatment of cancer (tumor) with an agent that inhibits hepatocyte growth factor together with an agent that blocks another cellular signaling pathway.

### BACKGROUND OF THE INVENTION

Human Hepatocyte Growth Factor (HGF) is a multifunctional heterodimeric polypeptide produced by mesenchymal cells. HGF has been shown to stimulate angiogenesis, morphogenesis and motogenesis, as well as the growth and scattering of various cell types (Bussolino et al., J. Cell. Biol. 119: 629, 1992; Zarnegar and Michalopoulos, J. Cell. Biol. 129:1177, 1995; Matsumoto et al, Ciba. Found. Symp. 212:198, 1997; Birchmeier and Gherardi, Trends Cell. Biol. 8:404, 1998; Xin et al., Am. J. pathol. 158:1111, 2001). The pleiotropic activities of HGF are mediated through its receptor, a transmembrane tyrosine kinase encoded by the proto-oncogene c-Met. In addition to regulating a variety of normal cellular functions, HGF and its receptor c-Met have been shown to be involved in the initiation, invasion and metastasis of tumors (Jeffers et al., J. Mol. Med. 74:505, 1996; Comoglio and Trusolino, J. Clin. Invest. 109:857, 2002). HGF/c-Met are coexpressed, often over-expressed, on various human solid tumors including tumors derived from lung, colon, rectum, stomach, kidney, ovary, skin, multiple myeloma and thyroid tissue (Prat et al., Int. J. Cancer 49:323, 1991; Chan et al., Oncogene 2:593, 1988; Weidner et al., Am. J. Respir. Cell Mol. Biol, 8:229, 1993; Derksen et al., Blood 99:1405, 2002). HGF acts as an autocrine (Rong et al., Proc. Natl. Acad. Sci. USA 91:4731, 1994; Koochekpour et al., Cancer Res. 57:5391, 1997) and paracrine growth factor (Weidner et al., Am. J. Respir. Cell Mol. Biol. 8:229, 1993) and anti-apoptotic regulator (Gao et al., J. Biol. Chem. 276:47257, 2001) for these tumors.

HGF is a 102 kDa protein with sequence and structural similarity to plasminogen and other enzymes of blood coagulation (Nakamura et al., Nature 342:440, 1989; Weidner et al., Am. J. Respir. Cell. Mol. Biol. 8:229, 1993, each of which is incorporated herein by reference). Human HGF is synthesized as a 728 amino acid precursor (preproHGF), which undergoes intracellular cleavage to an inactive, single chain form (proHGF) (Nakamura et al., Nature 342:440, 1989: Rosen et al., J. Cell. Biol. 127:1783, 1994). Upon extracellular secretion, proHGF is cleaved to yield the biologically active disulfide-linked heterodimeric molecule composed of an α-subunit and β-subunit (Nakamura et al., Nature 342:440, 1989; Naldini et al., EMBO J. 11:4825, 1992). The α-subunit contains 440 residues (69 kDa with glycosylation), consisting of the N-terminal hairpin domain and four kringle domains. The β-subunit contains 234 residues (34 kDa) and has a serine protease-like domain, which lacks proteolytic activity. Cleavage of HGF is required for receptor activation, but not for receptor binding (Hartmann et al., Proc. Natl. Acad. Sci. USA 89:11574, 1992; Lokker et al., J. Biol. Chem. 288:17145, 1992). HGF contains 4 putative N-glycosylation sites, 1 in the α-subunit and 3 in the β-subunit. HGF has 2 unique cell specific binding sites: a high affinity (Kd=2×10⁻¹⁰ M) binding site for the c-Met receptor and a low affinity (Kd=10⁻⁹ M) binding site for heparin sulfate proteoglycans (HSPG), which are present on the cell surface and extracellular matrix (Naldinl et al., Oncogene 6:501, 1991; Bardelii et al., J. Biotechnol. 37:109, 1994; Sakata et al., J. Biol. Chem., 272:9457, 1997).

c-Met is a member of the class IV protein tyrosine kinase receptor family. The full length c-Met gene was cloned and identified as the c-Met proto-oncogene (Cooper et al., Nature 311:29, 1984; Park et al., Proc. Natl. Acad. Sci. USA 84:6379, 1987). The c-Met receptor is initially synthesized as a single chain, partially glycosylated precursor, p170^{(MET)} (Park et al., Proc. Natl. Acad. Sci. USA 84:6379, 1987; Giordano et al., Nature 339:155, 1989; Giordano et al., Oncogene 4:1383, 1989; Bardelli et al., J. Biotechnol. 37:109, 1994). Upon further glycosylation, the protein is proteolytically cleaved into a heterodimeric 190 kDa mature protein (1385 amino acids), consisting of the 50 kDa α-subunit (residues 1-307) and the 145 kDa β-subunit. The cytoplasmic tyrosine kinase domain of the β-subunit is involved in signal transduction.

Several different approaches have been investigated to obtain HGF inhibitors, i.e. antagonists. Such inhibitors include truncated HGF proteins such as NK1 (N terminal domain plus kringle domain 1: Lokker et al., J. Biol. Chem. 268:17145, 1993); NK2 (N terminal domain plus kringle domains 1 and 2: Chan et al., Science 254:1382, 1991); and NK4 (N-terminal domain plus four kringle domains), which was shown to partially inhibit the primary growth and metastasis of murine lung tumor LLC in a nude mouse model (Kuba et al., Cancer Res. 60:6737, 2000).

As another approach, Dodge (Master's Thesis, San Francisco State University, 1998) generated antagonist anti-c-Met monoclonal antibodies (mAbs). One mAb, 5D5, exhibited strong antagonistic activity in ELISA, but induced a proliferative response of c-Met-expressing BAF-3 cells, presumably due to dimerization of the membrane receptors. For this reason, a single domain form of the anti-c-Met mAb 5D5 has been developed as an antagonist (Nguyen et al., Cancer Gene Ther, 10:840, 2003).

Cao et al., Proc. Natl. Acad. Sci. USA 98:7443, 2001, reported that the administration of a cocktail of three anti-HGF mAbs, which were selected based upon their ability to inhibit the scattering activity of HGF in vitro, were able to inhibit the growth of human tumors in the xenograft nude mouse model.

More recently, several neutralizing (inhibitory) anti-HGF mAbs have been reported including L2G7 (Kim et al., Clin Cancer Res 12:1292, 2006, WO 2005/107800 (Patent literature 1), and USP 7220410 (Patent literature 2)), HuL2G7 (WO 2007/115049 (Patent literature 3)), the human mAbs described in WO 2005/17107 (Patent literature 4), and the HGF binding proteins described in WO 2007/143090 (Patent literature 5) or WO 2007/143098 (Patent literature 6). It has also been reported that the anti-HGF mAb L2G7, when administered systemically, can strongly inhibit growth or even induce regression of orthotopic (intracranial) glioma xenografts and prolong animal survival (Kim et al., op. cit and WO 2006/130773 (Patent literature 7)).

Epidermal growth factor (EGF) is a widely distributed growth factor that in cancer (tumor), can stimulate cancer-cell proliferation, block apoptosis, activate invasion and metastasis, and stimulate angiogenesis (Citri et al., Nat Rev. Mol. Cell. Biol. 7:505, 2006; Hynes et al., Nat Rev. Cancer 5:341, 2005). The EGF receptor (EGFR or ErbB) is a transmembrane, tyrosine kinase receptor that belongs to a family of four related receptors. The majority of human epithelial cancers are marked by functional activation of growth factors and receptors of this family (Ciardiello et al., New Eng. J. Med. 358: 1160, 2008) so that EGF and EGFR are natural targets for cancer therapy. Activation of EGFR is commonly associated with mutations, for example, of exons 19 and 21 in some lung cancers, or deletion of exons 2-7 to form EGF receptor variant III (EGFRvIII) in many gliomas (Rosell et al., Clin. Cancer Res. 12:7222, 2006; Ji et al., Proc. Natl. Acad. Sci. USA 103:7817, 2006).

Four inhibitors of the EGF/EGFR pathway have been approved for marketing as drugs: Erbitux® (cetuximab, a chimeric anti-EGFR mAb) for colon cancer and squamous-cell cancer of the head and neck cancer, Vectibix® (panitumumab, a human anti-EGFR mAb) for colon cancer; Tarceva® (erlotinib) and Iressa® (gefitinib), both small molecule inhibitors of the tyrosine kinase activity of EGFR, with Tarceva for the treatment of non-small-cell lung cancer and pancreatic cancer and Iressa for the treatment of non-small-cell lung cancer in special circumstances. However, cancer cells can rapidly switch their dependence from EGFR to c-Met (RTK Switching; Stommel et al., Science 318:287, 2007), and EGFR-dependent tumors can develop resistance to the EGFR inhibitors erlotinib and gefitinib inhibitors by amplification of c-Met (Bean et al., Proc. Natl. Acad, Sci. USA 104:20932, 2007).

Petrelli et al., Proc. Natl. Acad, Sci. USA 103:5090, 2006 (Non patent literature 1) discloses the possibility of antitumor effect caused by anti-c-Met antibody. Jun et al., Clin. Cancer Res. 13:6735, 2007 (Non patent literature 2) discloses that combination data of anti-human HGF antibody and Temozolomide.

WO 2009/126840 (Patent literature 8) discloses a method of treating cancer by administering to a patient an inhibitor of HGF and an inhibitor of the Hedgehog signaling pathway.

WO 2009/126834 (Patent literature 9) discloses a method of treating cancer by administering to a patient a first agent that inhibits HGF in combination with a second agent that inhibits a signaling pathway other than the one stimulated by HGF (the HGF/c-Met pathway).

WO 2009/126842 (Patent literature 10) discloses a method of treating cancer by administering to a patient an inhibitor of Hepatocyte Growth Factor and an agonist of PTEN.

### [Citation List]

### [Patent Literature]

### [PTL 1] WO 2005/107800

[PTL 2] U.S. Pat. No. 7,2204,10

### [PTL 3] WO 2007/115049

### [PTL 4] WO 2005/17107

### [PTL 5] WO 2007/143090

### [PTL 6] WO 2007/143098

### [PTL 7] WO 2006/130773

### [PTL 8] WO 2009/126840

### [PTL 9] WO 2009/126834

### [PTL 10] WO 2009/126842

### [Non Patent Literature]

[NPL 1] Proc. Natl. Acad, Sci. USA 103:5090, 2006
[NPL 2] Clin. Cancer Res. 13:6735, 2007

### BRIEF SUMMARY OF THE INVENTION

The invention provides a method of treating cancer (tumor) by administering to a patient in need of such treatment a first agent that inhibits Hepatocyte Growth Factor (HGF) in combination with a second agent that inhibits a signaling pathway other than the one stimulated by HGF (the HGF/c-Met pathway). In a preferred embodiment, the first agent is a monoclonal antibody (mAb) that binds to and neutralizes HGF. Chimeric, human and humanized anti-HGF mAbs are especially preferred, particularly humanized L2G7. In some embodiments, the second agent is an inhibitor of epidermal growth factor (EGF), for example a mAb that binds to the EGF receptor, thereby inhibiting binding of EGF, such as cetuximab or panitumumab; or alternatively a small molecule inhibitor of the EGF pathway such as erlotinib or gefitinib. The second agent is also a nucleoside analogue such as gemcitabine, or an inhibitor of platelet-derived growth factor receptor (PDGFR), vascular endothelial growth factor receptor (VEGFR), c-KIT kinase, or FMS-like tyrosine kinase 3 (FLT3), such as sunitinib. The method is especially preferred for treating stomach (gastric), pancreas, lung, colon, head and neck cancer and brain tumors such as glioma.

The present invention provides the following:

[1] a method of treating a gastric tumor in a patient comprising administering to the patient a first agent that is a monoclonal antibody (mAb) that binds to and neutralizes human Hepatocyte Growth Factor (HGF) in combination with a second agent that is an EGFR antagonist,
[2] the method described in [1] above, wherein the EGFR antagonist is erlotinib,
[3] the method described in [1] above, wherein the mAb is chimeric, humanized or human and the mAb competes for binding to human HGF with an antibody produced by hybridoma ATCC Number PTA-5162,
[4] the method described in [1] above, wherein the mAb is a humanized L2G7,
[5] the method described in [1] above, wherein the first agent is a humanized L2G7 and the second agent is erlotinib,
[6] a pharmaceutical agent for treating a gastric tumor, comprising (1) a monoclonal antibody (mAb) that binds to and neutralizes human Hepatocyte Growth Factor (HGF), in combination with (2) an EGFR antagonist,
[7] use of (1) a monoclonal antibody (mAb) that binds to and neutralizes human Hepatocyte Growth Factor (HGF), in combination with (2) an EGFR antagonist, for the production of an agent for the treatment of a gastric tumor,
[8] a method of treating a pancreatic tumor in a patient comprising administering to the patient a first agent that is a monoclonal antibody (mAb) that binds to and neutralizes human Hepatocyte Growth Factor (HGF) in combination with a second agent that is a nucleoside analogue,
[9] the method described in [8] above, wherein the nucleoside analogue is gemcitabine,
[10] the method described in [8] above, wherein the mAb is chimeric, humanized or human and the mAb competes for binding to human HGF with an antibody produced by hybridoma ATCC Number PTA-5162,
[11] the method described in [8] above, wherein the mAb is a humanized L2G7,
[12] the method described in [8] above, wherein the first agent is a humanized L2G7 and the second agent is gemcitabine,
[13] a pharmaceutical agent for treating a pancreatic tumor, comprising (1) a monoclonal antibody (mAb) that binds to and neutralizes human Hepatocyte Growth Factor (HGF), in combination with (2) a nucleoside analogue,
[14] use of (1) a monoclonal antibody (mAb) that binds to and neutralizes human Hepatocyte Growth Factor (HGF), in combination with (2) a nucleoside analogue, for the production of an agent for the treatment of a pancreatic tumor,
[15] a method of treating a pancreatic tumor in a patient comprising administering to the patient a first agent that is a monoclonal antibody (mAb) that binds to and neutralizes human Hepatocyte Growth Factor (HGF) in combination with a second agent that is an inhibitor of platelet-derived growth factor receptor (PDGFR), vascular endothelial growth factor receptor (VEGFR), c-KIT kinase, or FMS-like tyrosine kinase 3 (FLT3),
[16] the method described in [15] above, wherein the inhibitor is sunitinib,
[17] the method described in [15] above, wherein the mAb is chimeric, humanized or human and the mAb competes for binding to human HGF with an antibody produced by hybridoma ATCC Number PTA-5162,
[18] the method described in [15] above, wherein the mAb is a humanized L2G7,
[19] the method described in [15] above, wherein the first agent is a humanized L2G7 and the second agent is sunitinib,
[20] a pharmaceutical agent for treating a pancreatic tumor, comprising (1) a monoclonal antibody (mAb) that binds to and neutralizes human Hepatocyte Growth Factor (HGF), in combination with (2) an inhibitor of platelet-derived growth factor receptor (PDGFR), vascular endothelial growth factor receptor (VEGFR), c-KIT kinase, or FMS-like tyrosine kinase 3 (FLT3),
[21] use of (1) a monoclonal antibody (mAb) that binds to and neutralizes human Hepatocyte Growth Factor (HGF), in combination with (2) an inhibitor of platelet-derived growth factor receptor (PDGFR), vascular endothelial growth factor receptor (VEGFR), c-KIT kinase, or FMS-like tyrosine kinase 3 (FLT3), for the production of an agent for the treatment of a pancreatic tumor,
[22] the method described in [1] above, wherein the mAb inhibits HGF-induced proliferation of IM95m human gastric tumor cells,
[23] the method described in [1] above, wherein the mAb inhibits growth of IM95m human gastric tumor xenograft in a mouse,
[24] the method described in [8] above, wherein the mAb inhibits HGF-induced proliferation of KP-4 human pancreatic tumor cells,
[25] the method described in [8] above, wherein the mAb inhibits growth of KP-4 human pancreatic tumor xenograft in a mouse,
[26] the method described in [15] above, wherein the mAb inhibits HGF-induced proliferation of KP-4 human pancreatic tumor cells, and
[27] the method described in [15] above, wherein the mAb inhibits growth of KP-4 human pancreatic tumor xenograft in a mouse.

### BRIEF DESCRIPTION OF THE DRAWINGS

FIG. 1. (A) Combination effects of humanized L2G7 (HuL2G7) and erlotinib in gastric cancer (tumor) IM95m xenograft model. Treatment was started from 53 days after inoculation. Mice treated with relevant vehicles (i.v. and/or p.o.), HuL2G7 (10 mg/kg/week, i.v., on days 53, 60, 67, and 74), erlotinib (25 mg/kg, p.o., qd×28 doses) or the combined regimen for 28 days. Data represents mean±SD (n=6). *p≦0.025 by one-tailed Dunnett's test (v.s. vehicle). (B) Comparison of differential tumor volumes of the mice group treated with HuL2G7, erlotinib, and a combination of the two. Tumor volume of each mouse on days 53 was subtracted from the volume on days 81. Data represents mean±SD (n=6). # p≦0.05 by Wilcoxon signed-rank test (v.s. combo).

FIG. 2. (A) Combination effects of HuL2G7 and gemcitabine in pancreatic cancer KP-4 xenograft model. Treatment was started from 11 days after inoculation. Mice treated with relevant vehicles (i.v. and/or i.p.), HuL2G7 (10 mg/kg/week, i.v., on days 11, 15, 18, 22, and 25), gemcitabine hydrochloride (100 mg/kg, i.p., on days 11, 18, and 25) or the combined regimen for 15 days. Data represents mean±SD (n=8). *p≦0.025 by one-tailed Steel's test (v.s. vehicle). (B) Comparison of differential tumor volumes of the mice group treated with HuL2G7, gemcitabine, and a combination of the two. Tumor volume of each mouse on days 11 was subtracted from the volume on days 29. Data represents mean±SD (n=8). # p≦0.05 by Welch's t-test (v.s. combo).

FIG. 3. (A) Combination effects of HuL2G7 and sunitinib in pancreatic cancer KP-4 xenograft model. Treatment was started from 11 days after inoculation. Mice treated with relevant vehicles (i.v. and/or p.o.), HuL2G7 (10 mg/kg/twice a weekly, i.v., on days 11, 15, 18, 22, and 25), sunitinib malate (40 mg/kg, p.o., on days 11-25) or the combined regimen for 15 days. Data represents mean±SD (n=8). *p≦0.025 by one-tailed Steel's test (v.s. vehicle). (B) Comparison of differential tumor volumes of the mice group treated with HuL2G7, sunitinib, and a combination of the two. Tumor volume of each mouse on days 11 was subtracted from the volume on days 29. Data represents mean±SD (n=8). # p≦0.05 by Student's t-test (v.s. combo).

### DETAILED DESCRIPTION OF THE INVENTION

The invention provides a method of treating cancer (tumor) by administering to a patient in need of such treatment a first agent that inhibits the activity of Hepatocyte Growth Factor (HGF), i.e., an HGF antagonist or c-Met antagonist, in combination with (i.e., together with) a second agent that inhibits a cellular signaling pathway other than the one stimulated by HGF (the HGF/c-Met pathway). In many embodiments, the first agent and/or the second agent is a monoclonal antibody (mAb).

### 1. Antibodies

Antibodies are very large, complex molecules (molecular weight of −150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions fold up together in 3-dimensional space to form a variable region that binds to the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-D space to form the actual antibody binding site which locks onto the target antigen. The position and length of the CDRs have been precisely defined (Kabat, E. et al., Sequences of Proteins of Immunological Interest, U.S. Department of Health and Human Services, 1983, 1987). The part of a variable region not contained in the CDRs is called the framework, which forms the environment for the CDRs.

A monoclonal antibody (mAB) is a single molecular species of antibody and therefore does not encompass polyclonal antibodies produced by injecting an animal (such as a rodent, rabbit or goat) with an antigen, and extracting serum from the animal. A humanized antibody is a genetically engineered (monoclonal) antibody in which the CDRs from a mouse antibody (“donor antibody”, which can also be rat, hamster or other similar species) are grafted onto a. human antibody (“acceptor antibody”). Humanized antibodies can also be made with less than the complete CDRs from a mouse antibody (e.g., Pascalls et al., J. Immunol. 169:3076, 2002). Thus, a humanized antibody is an antibody having CDRs from a donor antibody and variable region frameworks and constant regions from human antibodies. The light and heavy chain acceptor frameworks may be form the same or different human antibodies and may each be a composite of two or more human antibody frameworks; or alternatively may be a consensus sequence of a set of human frameworks (e.g., a subgroup of human antibodies as defined in Kabat et al, op. cit.), i.e., a sequence having the most commonly occurring amino acid in the set at each position. In addition, in order to retain high binding affinity, at least one of two additional structural elements can be employed. See, U.S. Pat. Nos. 5,530,101 and 5,585,089, each of which is incorporated herein by reference, which provide detailed instructions for construction of humanized antibodies.

In the first structural element, the framework of the heavy chain variable region of the humanized antibody is chosen to have maximal sequence identity (between 65% and 95%) with the framework of the heavy chain variable region of the donor antibody, by suitably selecting the acceptor antibody from among the many known human antibodies. Sequence identity is determined when antibody sequences being compared are aligned according to the Kabat numbering convention. In the second structural element, in constructing the humanized antibody, selected amino acids in the framework of the human acceptor antibody (outside the CDRs) are replaced with corresponding amino acids from the donor antibody, in accordance with specified rules. Specifically, the amino acids to be replaced in the framework are chosen on the basis of their ability to interact with the CDRs. For example, the replaced amino acids can be adjacent to a CDR in the donor antibody sequence or within 4-6 angstroms of a CDR in the humanized antibody as measured in 3-dimensional space.

A chimeric antibody is an antibody in which the variable region of a mouse (or other rodent) antibody is combined with the constant region of a human antibody; their construction by means of genetic engineering is well-known. Such antibodies retain the binding specificity of the mouse antibody, while being about two-thirds human. The proportion of nonhuman sequence present in mouse, chimeric and humanized antibodies suggests that the immunogenicity of chimeric antibodies is intermediate between mouse and humanized antibodies. Other types of genetically engineered antibodies that may have reduced immunogenicity relative to mouse antibodies include human antibodies made using phage display methods (Dower et al, WO 91/17271; McCafferty et al., WO 92/001047; Winter, WO 92/20791; and Winter, FEBS Lett 23:92. 1998, each of which is incorporation herein by reference) or using transgenic. animals (Lonberg et. al., WO 93/12227; Kucherlapati WO 91/10741, each of which is incorporated herein by reference).

As used herein, the term “human-like” antibody refers to a mAb in which a substantial portion of the amino acid sequence of one or both chains (e.g., about 50% or more) originates from human immunoglobulin genes. Hence, human-like antibodies encompass but are not limited to chimeric, humanized and human antibodies. As used herein, a “reduced-immunogenicity” antibody is one expected to have significantly less immunogenicity than a mouse antibody when administered to human patients. Such antibodies encompass chimeric, humanized and human antibodies as well as antibodies made by replacing specific amino acids in mouse antibodies that may contribute to B- or T-cell epitopes, for example exposed residues (Padlan, Mol. Immunol. 28:489, 1991). As used herein, a “genetically engineered” antibody is one for which the genes have been constructed or put in an unnatural environment (e.g., human genes in a mouse or on a bacteriophage) with the help of recombinant DNA techniques, and would therefore, e.g., not encompass a mouse mAb made with conventional hybridoma technology.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 50% but preferably 75%, 90% or even 99% as measured in a competitive binding assay compared to a control lacking the competing antibody (see, e.g., Junghans et al., Cancer Res. 50:1495, 1990, which is incorporated herein by reference). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

### 2. Antibodies for Use in the Invention

The antibody used in the invention is a monoclonal antibody that binds to and neutralizes human HGF (i.e., an anti-HGF mAb). Here, to neutralize human HGF means to inhibit one or more biological activities of human HGF partially or completely. Among the biological properties of HGF that a neutralizing antibody may inhibit are the ability of HGF to bind to its c-Met receptor, to cause the scattering of certain cell lines such as Madin-Darby canine kidney (MDCK) cells: to stimulate proliferation of (i.e., be mitogenic for) certain cells including hepatocytes, Mv 1 Lu mink lung epithelial cells, and various human tumor cells; or to stimulate angiogenesis, for example as measured by stimulation of human vascular endothelial cell (HUVEC) proliferation or tube formation or by induction of blood vessels when applied to the chick embryo chorioallantoic membrane (CAM). Antibodies for use in the invention preferably bind to human HGF, i.e., to the protein encoded by the GenBank sequence with Accession number D90334.

A neutralizing anti-HGF mAb is preferred for use as the first agent in the invention and, at a concentration of, e.g., 0.01, 0.1, 0.5, 1, 2, 5, 10, 20 or 50 μg/ml, inhibits a biological function of HGF (e.g., stimulation of proliferation or scattering) by about at least 50% but preferably 75%, more preferably by 90% or 95% or even 99%, and most preferably approximately 100% (essentially completely) as assayed by methods known in the art. Inhibition is considered complete if the level of activity is within the margin of error for a negative control lacking HGF. Typically, the extent of inhibition is measured when the amount of HGF used is just sufficient to fully stimulate the biological activity, or is 0.05, 0.1, 0.5, 1, 3 or 10 μg/ml. Preferably, at least 50%, 75%, 90%, or 95% or essentially complete inhibition is achieved when the molar ratio of antibody to HGF is 0.5×, 1×, 2× 3×, 5× or 10×. Preferably, the mAb is neutralizing, i.e., inhibits the biological activity when used as a single agent, but optionally 2 mAbs can be used together to give inhibition. Most preferably, the mAb neutralizes not just one but several of the biological activities listed above; for purposes herein, an anti-HGF mAb that used as a single agent neutralizes all the biological activities of HGF is called “fully neutralizing”, and such mAbs are most preferable. Anti-HGF mAbs for use in the invention are preferably specific for HGF, that is they do not bind, or only bind to a much lesser extent (e.g., Ka at least ten-fold less), proteins that are related to HGF such as fibroblast growth factor (FGF) and vascular endothelial growth factor (VEGF). Preferred antibodies lack agonistic activity toward HGF. That is, the antibodies block interaction of HGF with c-Met without stimulating cells bearing HGF directly. Anti-HGF mAbs for use in the invention typically have a binding affinity (Ka) for HGF of at least 10⁷ M⁻¹ but preferably 10⁸ M⁻¹ or higher, and most preferably 10⁹ M⁻¹ or higher or even 10¹⁰ M⁻¹ or higher.

mAbs for use in the invention include antibodies in their natural tetrameric form (2 light chains and 2 heavy chains) and may be of any of the known isotypes IgG, IgA, IgM, IgD and IgE and their subtypes, i.e., human IgG1, IgG2, IgG3, IgG4 and mouse IgG1, IgG2a, IgG2b, and IgG3. The mAbs are also meant to include fragments of antibodies such as Fv, Fab and F(ab′)₂: bifunctional hybrid antibodies (e.g., Lanzavecchia et al., Eur. J. Immunol. 17:105, 1987), single-chain antibodies (Huston et al., Proc. Natl. Acad. Sci. USA 85:5879, 1988; Bird et al., Science 242:423, 1988); and antibodies with altered constant regions (e.g. U.S. Pat. No. 5,624,821). The mAbs may be of animal (e.g., mouse, rat, hamster or chicken) origin, or they may be genetically engineered. Rodent mAbs are made by standard methods well-known in the art, comprising multiple immunization with HGF in appropriate adjuvant i.p., i.v., or into the footpad, followed by extraction of spleen or lymph node cells and fusion with a suitable immortalized cell line, and then selection for hybridomas that produce antibody binding to HGF, e.g., see under examples. Chimeric and humanized mAbs, made by art-known methods mentioned supra, are preferred for use in the invention. Human antibodies made, e.g., by phage display or transgenic mice methods are also preferred (see e.g., Dower et al McCafferty et al., Winter, Lonberg et al, Kucherlapati, supra). More generally, human-like, reduced immunogenicity and genetically engineered antibodies as defined herein are all preferred.

The neutralizing anti-HGF mAb L2G7 (which is produced by a hybridoma deposited at the American Type Culture Collection under ATCC Number PTA-5162 according to the Budapest treaty) as described in Kim et al., Clin Cancer Res 12:1292, 2006 and U.S. Pat. No. 7,220,410 and particularly its chimeric and humanized forms such as HuL2G7, as described in WO 2007/115049 A2, are especially preferred as the first agent in the invention. Neutralizing mAbs with the same or overlapping epitope as L2G7 and/or that compete with L2G7 for binding to HGF are also preferred. mAbs that are 90%, 95% or 99% identical to EG7 in amino acid sequence, when aligned according to the Kabat numbering convention, at least in the CDRs, and maintain its functional properties, or which differ from it by a small number of functionally inconsequential amino acid substitutions (e.g., conservative substitutions), deletions, or insertions can also be used in the invention.

Also preferred for use as the first agent in the invention are the anti-HGF mAbs described in WO 2005/017107 A2, whether explicitly by name or sequence or implicitly by description or relation to explicitly described mAbs. Especially preferred mAbs are those produced by the hybridomas designated therein as 1.24.1, 1.29.1, 1.60.1, 1.61.3, 1.74.3, 1.75.1, 2.4.4, 2.12.1, 2.40.1 and 3.10.1, and respectively defined by their heavy and light chain variable region sequences provided by SEQ ID NO's 24-43, with 2.12.1 being most preferred; mAbs possessing the same respective CDRs as any of these listed mAbs: mAbs having light and heavy chain variable regions that are at least 90%, 95% or 99% identical to the respective variable regions of these listed mAbs or differing from them only by inconsequential amino acid substitutions, deletions or insertions; mAbs binding to the same epitope of HGF as any of these listed mAbs, and all mAbs encompassed by claims 1 through 94 therein.

Alternatively, any of the HGF binding proteins described in WO 2007/143090 A2 or WO 2007/143098 A2 may be used as the first agent in the invention.

Native mAbs for use in the invention may be produced from their hybridomas. Genetically engineered mAbs, e.g., chimeric or humanized mAbs, may be expressed by a variety of art-known methods. For example, genes encoding their light and heavy chain V regions may be synthesized from overlapping oligonucleotides and inserted together with available C regions into expression vectors (e.g., commercially available from Invitrogen) that provide the necessary regulatory regions, e.g., promoters, enhancers, poly A sites, etc. Use of the CMV promoter-enhancer is preferred. The expression vectors may then be transfected using various well-known methods such as lipofection or electroporation into a variety of mammalian cell lines such as CHO or non-producing myelomas including Sp2/0 and NS0, and cells expressing the antibodies selected by appropriate antibiotic selection. See, e.g., U.S. Pat. No. 5,530,101. Larger amounts of antibody may be produced by growing the cells in commercially available bioreactors.

Once expressed, the mAbs for use in the invention may be purified according to standard procedures of the art such as microfiltration, ultrafiltration, protein A or D affinity chromatography, size exclusion chromatography, anion exchange chromatography, cation exchange chromatography and/or other forms of affinity chromatography based on organic dyes or the like. Substantially pure antibodies of at least about 90 or 95% homogeneity are preferred, and 98% or 99% or more homogeneity most preferred, for pharmaceutical uses. The mAbs are typically provided in a pharmaceutical formulation, i.e., in a physiologically acceptable carrier, optionally with excipients or stabilizers. Acceptable carriers, excipients or stabilizer are nontoxic to recipients at the dosages and concentrations employed, and include buffers such as phosphate, citrate, or acetate at a pH typically of 6.0 to 8.0, most often 6.0 to 7.0: salts such as sodium chloride, potassium chloride, etc. to make isotonic: antioxidants, preservatives, low molecular weight polypeptides, proteins, hydrophilic polymers such as polysorbate 80, amino acids, carbohydrates, chelating agents, sugars, and other standard ingredients known to those skilled in the art (Remington's Pharmaceutical Science 16^{th} edition, Osol, A. Ed. 1980). The mAb is typically present at a concentration of 1-100 mg/ml, e.g., 10 mg/ml. It will be understood that unless otherwise specified, by “patient” is meant a human patient, and therefore herein a mAb binding to any protein means a mAb binding to the respective human protein. The mAb used as the first agent in the present invention is most preferably chimeric, humanized or human mAb which competes for binding to human HGF with an antibody produced by hybridoma ATCC Number PTA-5162. Among all, a humanized L2G7 is preferred.

### 3. Other Agents for Use in the Invention

Besides anti-HGF mAbs, the first agent for use in the invention may be any other agent that inhibits HGF, i.e., inhibits its biological activity, and may therefore be called an HGF antagonist. Examples are soluble forms of c-Met (e.g., see Michiell et al., Cancer Cell 6:61, 2004) and a cocktail of several anti-HGF mAbs (Cao et al., Proc. Natl. Acad. Sci. USA 98:7443, 2001). As used herein the term “agent that inhibit HGF” or “HGF inhibitor” also includes an agent that interacts with the c-Met receptor of HGF so as to inhibit HGF signaling through c-Met; such an agent may also be called a c-Met inhibitor or antagonist. However, as used herein, inhibitors or antagonists of HGF or c-Met or the HGF/c-Met pathway are not meant to include agents that inhibit signaling events, such as activation of MAP kinase, that occur alter (i.e., downstream) of the HGF-c-Met interaction and activation of c-Met, and which the HGF/c-Met pathway shares with other ligand/receptor systems. A c-Met antagonist may function by binding to c-Met and competitively blocking binding of HGF or activation by HGF. Exemplary agents include truncated HGF proteins such as NK1, NK2, and NK4 (supra) and anti-c-Met mAbs. A preferred example is an anti-c-Met antibody that has been genetically engineered to have only one “arm”, i.e., binding domain, such as OA-5D5 (Martens et al., Clin. Cancer Res. 12:6144, 2006). Such agents may also be small molecule inhibitors of the tyrosine kinase activity of c-Met including SU5416 (Wang et al., J Hepatology 41:267.2004), and ARQ 197 being developed by ArQule, Inc. (Abstract Number 3525 at the 2007 Annual Meeting of the American Society of Clinical Oncology), which may be administered orally.

The second agent for use in the invention is any inhibitor of a cellular signaling pathway other than the HGF/c-Met pathway. Such an agent may bind to the ligand stimulating the pathway or to its receptor or to a downstream signaling molecule. The agent may be a protein such as a mAb, preferably a chimeric, humanized or human mAb, which binds to the ligand or receptor, or may be a small molecule (i.e., a compound having relatively low molecular weight, most often less than 500 or 600 or 1000 kDa). Proteins are typically administered parenterally, e.g. intravenously, whereas small molecules may be administered parenterally or orally. The ligand is often a cytokine or growth factor, whereas the receptor is often a tyrosine kinase, so that tyrosine kinase inhibitors are preferred as a second agent in the invention. For example, the second agent may be an agent that inhibits EGF, i.e., inhibits its biological activity. An “agent that inhibits EGF” or “EGF inhibitor” includes an agent that interacts with the EGFR so as to inhibit EGF signaling through EGFR; such an agent may also be called an EGFR inhibitor or antagonist.

The second agent for use in the invention is preferably an EGFR antagonist; a nucleoside analogue; or an inhibitor of platelet-derived growth factor receptor (PDGFR), vascular endothelial growth factor receptor (VEGFR), c-KIT kinase, or FMS-like tyrosine kinase 3 (FLT3). An EGFR antagonist may function by binding to EGFR and competitively blocking binding of EGF or activation by EGF, for example the anti-EGFR mAbs cetuximab and panitumumab, or by inhibiting the tyrosine kinase activity of EGFR, for example erlotinib and gefitinib. Among all, erlotinib is preferred. More generally, downstream signaling pathways that may be inhibited by the second agent in the invention include the RAS-RAF-MEK-APK pathway and the P13K-AKT pathway. Many other signaling pathways and their inhibitors are well known to those skilled in the art of cellular biology. The nucleoside analogues include gemcitabine, methotrexate, 5-fluorouracil, cytosine arabinoside, behenoyl cytosine arabinoside, tegafur, UFT, and the like. Among all, gemcitabine is preferred. The inhibitors of PDGFR, VEGFR, c-KIT kinase or FLT3 include sunitinib, sorafenib, motesanib, and the like. Among all, sunitinib is preferred.

### 4. Treatment Methods

The invention provides methods of treatment in which the indicated first and second agents are administered to patients having a cancer (therapeutic treatment) or at risk of occurrence or recurrence of a cancer (prophylactic treatment).

A mAb or other proteins used as a first or second agent in the methods of the invention can be administered to a patient by any suitable route, especially parenterally by intravenous (IV) infusion or bolus injection, intramuscularly or subcutaneously or intraperitoneally. IV infusion can be given over as little as 15 minutes, but more often for 30 minutes, 60 minutes, 90 minutes or even 2 or 3 hours. The agent can also be injected directly into the site of disease (e.g., the tumor itself; or the brain or its surrounding membranes or cerebrospinal fluid in the case of a brain tumor) or encapsulated into carrying agents such as liposomes. However, when treating brain tumors, systemic administration of the mAb, e.g., by IV infusion, is possible and even preferred (see WO 2006/130773 A2). The dose given to a patient having a cancer is sufficient to alleviate or at least partially arrest the cancer to be treated (“therapeutically effective dose”) and is sometimes 0.1 to 30 mg/kg body weight, for example 1, 2, 3, 4, 5 or 6 mg/kg, but may be as high as 10 mg/kg or even 15 or 20 or 30 mg/kg. A fixed unit dose may also be given, for example, 50, 100, 200, 500 or 1000 mg, or the dose may be based on the patient's surface area, e.g., 100 mg/m². Usually between 1 and 8 doses, (e.g., 1, 2, 3, 4, 5, 6, 7 or 8) are administered to treat cancer, but 10, 12, 20 or more doses may be given. The agent can be administered daily, biweekly, weekly, every other week, monthly or at some other interval, depending, e.g. on its half-life, for 1 week, 2 weeks, 4 weeks, 8 weeks, 3-6 months or longer, or until the disease progresses. Repeated courses of treatment are also possible, as is chronic administration.

When a small molecule is used as the first or second agent, it is typically administered more often, preferably once a day, but 2, 3, 4 or more times per day is also possible, as is every two days. Small molecule drugs are often taken orally but parenteral administration is also possible, e.g., by IV infusion or bolus injection or subcutaneously or intramuscularly. Doses of small molecule drugs are typically 10 to 1000 mg, with 100, 150, 200 or 250 mg very typical, with the optimal dose established in clinical trials. For either a protein or small molecule drug, a regime of a dosage and intervals of administration that alleviates or at least partially arrests the symptoms of a disease (biochemical, histologic and/or clinical), including its complications and intermediate pathological phenotypes in development of the disease is referred to as a therapeutically effective regime.

When a first agent (an anti-HGF mAb) is used in combination with a second agent (e.g., an EGF antagonist, a nucleoside analogue, or an inhibitor of platelet-derived growth factor receptor (PDGFR), vascular endothelial growth factor receptor (VEGFR), c-KIT kinase, or FMS-like tyrosine kinase 3(FLT3)), the combination may take place over any convenient timeframe. For example, each agent may be administered to a patient on the same day, and the agents may even be administered in the same intravenous infusion. However, the agents may also be administered on alternating days or alternating weeks, fortnights or months, and so on. In some methods, the respective agents are administered with sufficient proximity in time that the agents are simultaneously present (e.g., in the serum) at detectable levels in the patient being treated. In some methods, an entire course of treatment of one agent consisting of a number of doses over a time period (see above) is followed by a course of treatment of the other agent also consisting of a number of doses. In some methods, treatment with the agent administered second is begun if the patient has resistance or develops resistance to the agent administered initially. The patient may receive only a single course of treatment with each agent or multiple courses with one or both agents. Frequently, a recovery period of 1, 2 or several days or weeks is allowed between administration of the two agents if this is beneficial to the patient in the judgment of the attending physician. When a suitable treatment regimen has already been established for one of the agents, that regimen is preferably used when the agent is used in combination with the other. For example, Tarceva® (erlotinib) is taken as a 10 to 200 mg pill once a day (for example as a 25 mg, 100 mg or 150 mg pill once a day), and Iressa® (gefitinib) is taken as 250 mg tablet daily. Erbitux® (cetuximab) is administered as an IV infusion in an initial dose of 400 mg/m² followed by weekly 250 mg/m² doses, and Vectibix® (panitumumab) is administered as an IV infusion of 6 mg/kg every 2 weeks. Gemzar® (gemcitabine) is administered intravenously at 800 to 1250 mg/m² over 30 minutes (for example 1000 mg/m² over 30 minutes) on Days 1, 8, and 15 of each 28-day cycle. Sutent® (sunitinib) is taken as a 10 to 100 mg pill once a day (for example as a 25 mg or 50 mg capsules once a day) for 4 weeks followed by 2 weeks off. Typically, these agents are administered until the disease progresses.

The methods of the invention can also be used in prophylaxis of a patient at risk of cancer (tumor). Such patients include those having genetic susceptibility to cancer, patients who have undergone exposure to carcinogenic agents, such as radiation or toxins, and patients who have undergone previous treatment for cancer and are at risk of recurrence. A prophylactic dosage is an amount sufficient to eliminate at reduce the risk, lessen the severity, or delay the outset of the disease, including biochemical, histologic and/or clinical symptoms of the disease, its complications and intermediate pathological phenotypes presenting during development of the disease. Administration of a pharmaceutical composition in an amount and at intervals effective to effect one or more of these objects is referred to as a prophylactically effective regimen. The dosages and regimens disclosed above for therapeutic treatment can also be used for prophylactic treatment.

Types of cancer especially susceptible to treatment using the methods of the invention include solid tumors known or suspected to require angiogenesis or to be associated with elevated levels of HGF or c-Met, for example ovarian cancer, breast cancer, lung cancer (small cell or non-small cell), talon cancer, prostate cancer, pancreatic cancer (tumor), bladder cancer, cervical cancer, renal cancer, gastric cancer (tumor), liver cancer, head and neck tumors, mesothelioma, melanoma, and sarcomas, and brain tumors. Treatment can also be administered to patients having leukemias or lymphomas. The methods of the invention can also be used for treatment of brain tumor including meningiomas; gliomas including ependymomas, oligodendrogliomas, and all types of astrcytomas (low grade, anaplastic, and glioblastoma multiforme or simply glioblastoma); medullablastomas, gangliogliomas, schwannomas, chordomas; and brain tumor primarily of children including primitive neuroectodermal tumors. Both primary brain tumors (i.e., arising in the brain) and secondary or metastatic brain tumors can be treated by the methods of the invention. When the second agent is an EGF inhibitor, tumors known to be susceptible to one or more of the approved EGF inhibitor drugs are especially preferred, e.g., stomach (gastric), pancreas, lung, colon, head and neck, and brain cancer. Tumor types or individual tumors in which the EGFR is over-active, typically because of mutation (e.g., EGFRvIII) or amplification, are most preferred as the target of treatment. The methods of the present invention are particularly suitable for treatment of a gastric tumor when an EGFR antagonist such as erlotinib is used as the second agent. The methods of the present invention are particularly suitable for treatment of a pancreatic tumor when a nucleoside analogue such as gemcitabine is used as the second agent. Also, the methods of the present invention are particularly suitable for treatment of a pancreatic tumor when an inhibitor of PDGFR, VEGFR, c-KIT kinase or FLT3, such as sunitinib, is used as the second agent.

Because of the severity of cancer, several drugs to treat the disease are often given in combination. Hence, in a preferred embodiment of the present invention, the first agent (an anti-HGF mAb) and the second agent (e.g., an EGFR antagonist) are administered together with additional anti-cancer drugs. The first agent and second agent can be administered before, during or after the other anti-cancer drugs. For example, the first and second agents may be administered together with any one or more of the chemotherapeutic drugs known to those of skill in the art of oncology, for example alkylating agents such as carmstine, chlorambucil, cisplatin, carboplatin, oxaliplatin, procarbazine, and cyclophosphamide; antimetabolites such as fluorouracil, floxuridine, fludarabine, gemcitabine, methotrexate and hydroxyurea; natural products including plant alkaloids and antibiotics such as bleomycin, doxorubicin, daunorubicin, idarubicin, etoposide, mitomycin, mitioxantrone, vinblasine, vincristine, and Taxol (paclitaxel) or related compounds such as Taxotere®; the topoisomerase 1 inhibitor such as irinotecan; agents specifically approved for brain tumors including temozolomide and Gliadel® wafer containing carmustine; and inhibitors of tyrosine kinases such as Gleevec® and Sutent® (sunitinib malate); and all approved and experimental anti-cancer agents listed in WO 2005/017107 A2 (which is herein incorporated by reference). The first and second agents can be administered in combination with 1, 2, 3 or more of these other anti-cancer drugs used in a chemotherapeutic regimen. The other anti-cancer drugs can be used at the dosage known to be effective for the particular type of cancer to be treated. Moreover, the first and second agents can be administered together with any form of radiation therapy including external beam radiation, intensity modulated radiation therapy (IMRT) and any form of radiosurgery inducing Gamma Knife, Cyberknife, Linac, and interstitial radiation (e.g. implanted radioactive seeds, GliaSite balloon), and/or with surgery. Combination with radiation therapy can be especially appropriate for head and neck cancer and brain tumor. Other agents with which the first and second agents can be administered include biologics such as monoclonal antibodies, including Herceptin™ against the HER2 antigen and Avastin™ against VEGF.

The progression-free survival or overall survival time of patient with cancer (e.g., ovarian, prostate, breast, lung, colon, stomach (gastric), pancreas, kidney, head and neck, and brain, especially when relapsed or refractory) treated according to the method of the invention with the first and second agents may increase by at least 10%, 20%, 30% or 40% but preferably 50%, 60% to 70% or even 80%, 90%, 100% or longer, compared to patients treated similarly (e.g., with standard chemotherapy or without specific therapy) but without the first and second agents. The median progression-free survival or overall survival time may also be increased by at least 10 days, but preferably 30 days, 60 days, or 3, 4, 5 or 6 months or 1 year or longer by treatment according to the method of the invention. In addition or alternatively, treatment by the method of the invention may increase the complete response rate, partial response rate, or objective response rate (complete +partial) of patients by at least 10%, 20%, 30% or 40% but preferably 50%, 60% to 70% or even 80%, 90% or 100%. Moreover, when administering treatment with two agents, the regimes with which the respective agents are administered are combined in such a manner that each agent can make a contribution to the therapy, so treatment according to the invention with the first and second agents can increase progression-free or overall survival or increase the complete, partial or objective response rate by at least 10%, 20%, 30% or 40% but preferably 50%, 60% to 70% or even 80%, 90% or 100% compared to treatment with either agent without the other. Indeed, preferably treatment with the first and second agents is synergistic, i.e., better than additive. Optionally, treatment according to the method of the invention can inhibit tumor invasion, or metastasis.

Typically, in a clinical trial (e.g., a phase II, phase II/III or phase III trial), the aforementioned increases in median progression-free survival and/or response rate of the patients treated by the method of the invention together with a standard therapy (e.g., a chemotherapeutic regimen), relative to the control group of patients receiving the standard therapy alone, is statistically significant, for example at the p<0.05 or 0.01 or even 0.001 level. The complete and partial response rates can be determined by objective criteria commonly used in clinical trials for cancer, e.g., as listed or accepted by the National Cancer Institute and/or Food and Drug Administration.

### 5. Pharmaceutical Agents

The present invention provides a pharmaceutical agent for treating a cancer (tumor), comprising (1) the first agent (an anti-HGF mAb) in combination with (2) the second agent (e.g., an EGFR antagonist, a nucleoside analogue, or an inhibitor of PDGFR, VEGFR, c-KIT kinase or FLT3). Particularly, the pharmaceutical agent of the present invention is effective for treating a gastric tumor when an EGFR antagonist such as erlotinib is used as the second agent. The pharmaceutical agent of the present invention is effective for treating a pancreatic tumor when a nucleoside analogue such as gemcitabine is used as the second agent. Also the pharmaceutical agent of the present invention is effective for treating a pancreatic tumor when an inhibitor of PDGFR, VEGFR, c-KIT kinase or FLT3, such as sunitinib, is used as the second agent.

A pharmaceutical agent of the present invention has low toxicity, and can be safely administered orally or parenterally (e.g., subcutaneous, topical, rectal, intravenous administrations, etc.) to the patient (human patient). The pharmaceutical agent can be produced by mixing the first agent and the second agent, according to a method known per se, with a pharmacologically acceptable carrier to give pharmaceutical compositions, such as tablets (including sugar-coated tablet, film-coated tablet), powders, granules, capsules, solutions, emulsions, suspensions, injections, suppositories, sustained release preparations (e.g., sublingual tablet, microcapsule, etc.), plasters, orally disintegrating tablets, orally disintegrating films and the like.

As pharmacologically acceptable carriers usable for the production of the pharmaceutical agent of the present invention, various organic or inorganic carrier substances conventionally used as preparation materials can be mentioned. For example, suitable amounts of additives such as excipient, lubricant, binder and disintegrant for solid preparations, or solvent, solubilizing agent, suspending agent, isotonicity agent, buffer and soothing agent for liquid preparations, and where necessary, conventional preservative, antioxidant, coloring agent, sweetening agent, adsorbent, wetting agent and the like can be used appropriately.

When using the pharmaceutical agent of the present invention, the administration time of the first agent and the second agent is not restricted, and the first agent or a pharmaceutical composition thereof and the second agent or a pharmaceutical composition thereof can be administered to an administration subject simultaneously, or may be administered at different times. The dosage of the first agent or the second agent may be determined according to the administration amount clinically used, and can be appropriately selected depending on an administration subject, administration route, disease, combination and the like.

Examples of such administration mode include the following:

(1) administration of a single preparation obtained by simultaneously processing the first agent and the second agent,
(2) simultaneous administration of two kinds of preparations of the first agent and the second agent, which have been separately produced, by the same administration route, (3) administration of two kinds of preparations of the first agent and the second agent, which have been separately produced, by the same administration route in a staggered manner, (4) simultaneous administration of two kinds of preparations of the first agent and the second agent, which have been separately produced, by different administration routes, (5) administration of two kinds of preparations of the first agent and the second agent, which have been separately produced, by different administration routes in a staggered manner (for example, administration in the order of first agent and the second agent, or in the reverse order) and the like.

The amount ratio of the first agent to the second agent in the pharmaceutical agent of the present invention can be appropriately selected depending on an administration subject, administration route, diseases and the like.

For example, the content of the first agent in the pharmaceutical agent of the present invention varies depending on the form of a preparation, and usually from about 0.01 to 99.9 wt %, preferably from about 2 to 85 wt %, further preferably from about 9 to 29 wt %, based on the total amount of the first agent and the second agent.

While the content of the second agent in the pharmaceutical agent of the present invention varies depending on the form of a preparation, it is usually from about 0.01 to 99.9 wt %, preferably from about 2 to 95 wt %, further preferably from about 70 to 92 wt %, based on the total amount of the first agent and the second agent.

While the content of the additives such as carrier and the like in the pharmaceutical agent of the present invention varies depending on the form of a preparation, it is generally from about 1 to 99.9 wt %, preferably from about 5 to 90 wt %, based on the total amount of the first agent and the second agent.

While the compounding ratio of the first agent to the second agent in the pharmaceutical agent of the present invention can be appropriately selected depending on an administration subject, administration route, diseases and the like, it is usually from 1:0.1 to 1:100, preferably from 1:1 to 1:50, further preferably from 1:2.5 to 1:10.

The pharmaceutical agent of the present invention can also be used by mixing or combining with other anti-cancer drugs as described above, in appropriate amounts.

### EXAMPLES

### 1. Anti Tumor Efficacy of a Humanized L2G7 (HuL2G7) in Combination with Erlotinib in Human Gastric Cancer IM95m Tumor Xenograft Model

The combination effect of HuL2G7 with erlotinib was examined in human gastric cancer IM95m tumor xenograft model.

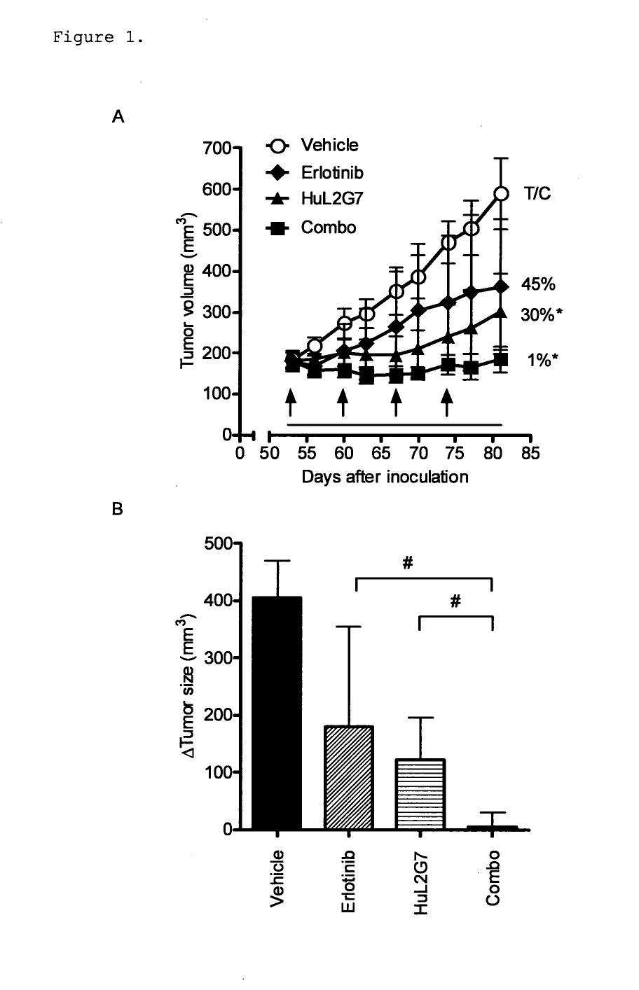

Human gastric cancer (tumor) cell line IM95m (JCRB1075.1) was obtained from Health Science Research Resources Bank (Osaka, Japan). The cells were proliferated in DMEM medium (Invitrogen) supplemented with 10% heat-inactivated fetal bovine serum (HyClone), 10 mg/L human insulin solution (SIGMA), 100 units/mL penicillin G and 100 μg/mL streptomycin (Wako Chemicals). The cells were cultured in tissue culture dishes in a humidified incubator at 37° C. in an atmosphere of 5% CO₂ and 95% air. Female severe combined immunodeficiency mice (C.B-17/Icr-scid/scid, 60 animals) were purchased from CLEA Japan (Tokyo, Japan). Mice were housed in a barrier facility with 12-h light/dark cycles and provided with food and tap water ad libitum and maintained for a week. IM95m cells (1×10⁷ in 100 μL) were suspended with 50% Matrigel (BD Biosciences) buffered with Hanks balanced salt solution (Invitrogen Corp, Carlsbad, Calif., USA) and inoculated subcutaneously into the right-flank of each mouse. After the tumor xenografts were established, at day 53 after inoculation, mice with tumors (approximately 150-230 mm³) were randomized into groups of 4 (n=6) based on tumor volumes so as to minimize variation in tumor volumes. Then each group was treated with vehicle (saline and 0.5% methyl cellulose solution), HuL2G7, erlotinib, or both drugs. HuL2G7 was administered intravenously and once-weekly with a dose of 10 mg/kg on days 53, 60, 67, and 74. Erlotinib was administered orally and once-daily at a dose of 25 mg/kg on days 53-80 as its 0.5% methyl cellulose suspension. Administration volume was 10 mL/kg. HuL2G7 was stored in a refrigerator and diluted in saline at room temperature just before injection. There were no abnormalities in general observations of all animals. Tumor volumes were assessed by bilateral vernier caliper measurement at days 53, 56, 60, 63, 67, 70, 74, 77, and 81 after inoculation and calculated using the formula, length×width^{2×}½, where length was taken to be the longest diameter across the tumor and width the corresponding perpendicular. Tumor growth was evaluated by the following formula: T/C (%)=ΔT/ΔC×100, where ΔT and ΔC are changes in tumor volume for each treated and vehicle control group. The T/C values of mice treated with HuL2G7 alone, erlotinib alone, and a combination of the two were 30, 45, and 1%, respectively (FIG. 1A). Significant difference of ΔT values between the mice group treated with a combination of the two drugs and the mice group treated with HuL2G7 alone or erlotinib alone was observed (p≦0.05) by Wilcoxon test (FIG. 1B). As a result, administration of HuL2G7 in combination with erlotinib exerted stronger antitumor activity than each single treatment in IM95m gastric cancer mice xenograft model.

### 2. Anti Tumor Efficacy of Humanized L2G7 (HuL2G7) in Combination with Gemcitabine in Human Pancreatic Cancer KP-4 Tumor Xenograft Model

The combination effect of HuL2G7 with gemcitabine was examined in human pancreatic cancer KP-4 tumor xenograft model.

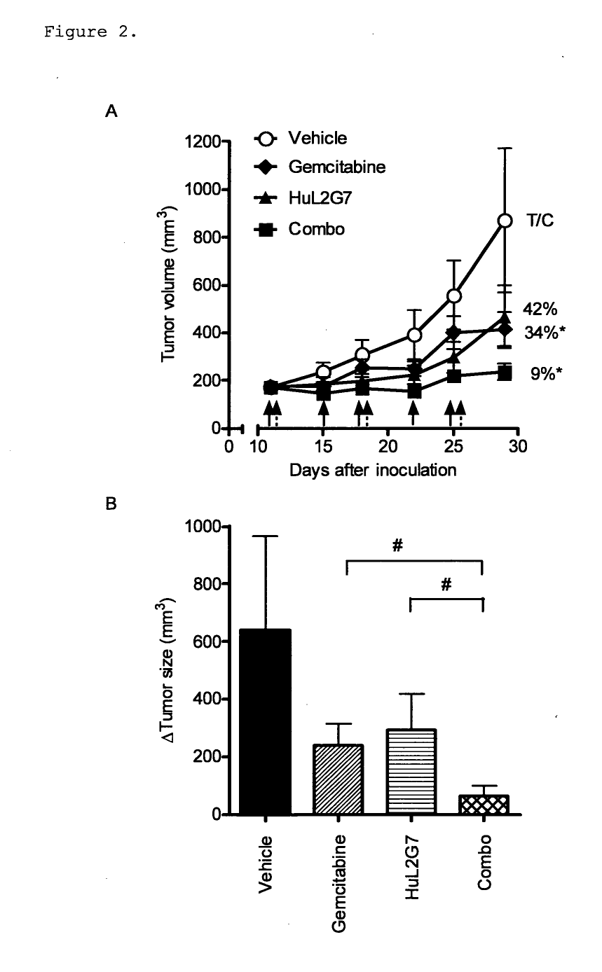

Human pancreatic cancer (tumor) line KP-4 (JCRB No.0182) was obtained from Health Science Research Resources Bank (Osaka, Japan). The cells were proliferated in RPMI medium (Invitrogen) supplemented with 10% heat-inactivated fetal bovine serum (HyClone) and 100 μg/mL streptomycin (Wako Chemicals). The cells were cultured in tissue culture dishes in a humidified incubator at 37° C. in an atmosphere of 5% CO₂ and 95% air. Female athymic nude mice (BALB/c-nu/nu, 50 animals) were purchased from Japan Clea (Tokyo, Japan). Mice were housed in a barrier facility with 12-h light/dark cycles and provided with food and tap water ad libitum and maintained for 13 days. KP-4 cells (5×10⁶ in 100 μL) were suspended with 50% Matrigel (BD Biosciences) buffered with Hanks balanced salt solution (Invitrogen Corp, Carlsbad, Calif., USA) and inoculated subcutaneously into the right-flank of each mouse. After the tumor xenografts were established, at day 11 after inoculation, mice with tumors (approximately 156-206 mm³) were randomized into groups of 4 (n=8) based on tumor volumes and body weights so as to minimize variation in tumor volumes and body weights. Then each group was treated with vehicle (saline), HuL2G7, gemcitabine or both drugs. HuL2G7 was, administered intravenously and twice-weekly with a dose of 10 mg/kg on days 11, 15, 18, 22, 25 and 29. Gemcitabine was administered intraperitoneal and once-weekly at a dose of 100 mg/kg on days 11, 18, 25 as its saline solution. Administration volume was 10 mL/kg. HuL2G7 was stored in a refrigerator and diluted in saline at room temperature just before injection. There were no abnormalities in general observations of all animals. Tumor volumes were assessed by bilateral vernier caliper measurement on days 11, 15, 18, 22, 25 and 29 after inoculation and calculated using the formula, length×width^{2×}½, where length was taken to be the longest diameter across the tumor and width the corresponding perpendicular. Tumor growth was evaluated by the following formula: T/C (%)=ΔT/ΔC×100, where ΔT and ΔC are changes in tumor volume for each treated and vehicle control group. The T/C values of mice treated with HuL2G7 alone, gemcitabine alone, and a combination of the two were 42, 34, and 9%, respectively (FIG. 2A). Significant difference of ΔT values between the mice group treated with a combination of the two drugs and the mice group treated with HuL2G7 alone or gemcitabine alone was observed (p<0.05) by one-tailed Student's T-test (FIG. 2B). As a result, administration of HuL2G7 in combination with gemcitabine exerted stronger antitumor activity than each single treatment in KP-4 pancreatic cancer mice xenograft model.

### 3. Anti Tumor Efficacy of Humanized L2G7 (HuL2G7) in Combination with Sunitinib in Human Pancreatic Cancer KP-4 Tumor Xenograft Model

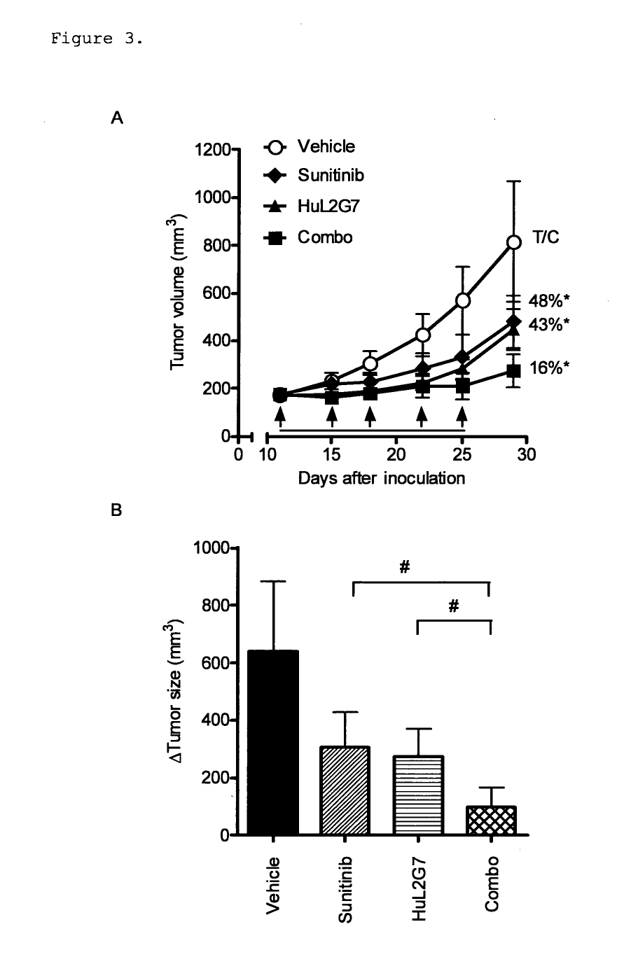

The combination effect of HuL2G7 with sunitinib was examined in human pancreatic cancer KP-4 tumor xenograft model. Female athymic nude mice (BALB/c-nu/nu, 50 animals) were purchased from Japan Clea (Tokyo, Japan). Mice were housed in a barrier facility with 12-h light/dark cycles and provided with food and tap water ad libitum and maintained for 10 days. KP-4 cells (5×10⁶ in 100 μL) were suspended with 50% Matrigel (BD Biosciences) buffered with Hanks balanced salt solution (Invitrogen Corp, Carlsbad, Calif., USA) and inoculated subcutaneously into the right-flank of each mouse. After the tumor xenografts were established, at day 11 after inoculation, mice with tumors (approximately 150-202 mm³) were randomized into groups of 4 (n=8) based on tumor volumes and body weights so as to minimize variation in tumor volumes and body weights. Then each group was treated with vehicle (saline and 0.5% methyl cellulose solution), HuL2G7, sunitinib or both drugs. HuL2G7 was administered intravenously and twice-weekly with a dose of 10 mg/kg on days 11, 15, 18, 22, 25 and 29. Sunitinib was administered orally and once-daily at a dose of 40 mg/kg on days 11-25 as its 0.5% methyl cellulose suspension. Administration volume was 10 mL/kg. HuL2G7 was stored in a refrigerator and diluted in saline at room temperature just before injection. There were no abnormalities in general observations of all animals. Tumor volumes were assessed by bilateral vernier caliper measurement on days 11, 15, 18, 22, 25 and 29 after inoculation and calculated using the formula, length×width^{2×}½, where length was taken to be the longest diameter across the tumor and width the corresponding perpendicular. Tumor growth was evaluated by the following formula: T/C (%)=ΔT/ΔC×100, where ΔT and ΔC are changes in tumor volume for each treated and vehicle control group. The T/C values of mice treated with HuL2G7 alone, sunitinib alone, and a combination of the two were 43, 48, and 16%, respectively (FIG. 3A). Significant difference of AT values between the mice group treated with a combination of the two drugs and the mice group treated with HuL2G7 alone or sunitinib alone was observed (p≦0.05) by one-tailed Student's T-test (FIG. 3B). As a result, administration of HuL2G7 in combination with sunitinib exerted stronger antitumor activity than each single treatment in KP-4 pancreatic cancer mice xenograft model.

This application is based on U.S. provisional application No. 61/202892 filed in United States (filing date; Apr. 17, 2009), the contents of which are hereby incorporated by reference.

1. A method of treating a gastric tumor in a patient comprising administering to the patient a first agent that is a monoclonal antibody (mAb) that binds and neutralizes human Hepatocyte Growth Factor (HGF) in combination with a second agent that is an EGFR antagonist.

2. The method of claim 1 wherein the EGFR antagonist is erlotinib.

3. The method of claim 1 wherein the mAb is chimeric, humanized or human and the mAb competes for binding to human HGF with an antibody produced by hybridoma ATCC Number PTA-5162.

4. The method of claim 1 wherein the mAb is a humanized L2G7.

5. The method of claim 1 wherein the first agent is a humanized L2G7 and the second agent is erlotinib.

6-7. (canceled)

8. A method of treating a pancreatic tumor in a patient comprising administering to the patient a first agent that is a monoclonal antibody (mAb) that binds and neutralizes human Hepatocyte Growth Factor (HGF) in combination with a second agent that is a nucleoside analogue.

9. The method of claim 8 wherein the nucleoside analogue is gemcitabine.

10. The method of claim 8 wherein the mAb is chimeric, humanized or human and the mAb competes for binding to human HGF with an antibody produced by hybridoma ATCC Number PTA-5162.

11. The method of claim 8 wherein the mAb is a humanized L2G7.

12. The method of claim 8 wherein the first agent is a humanized L2G7 and the second agent is gemcitabine.

13-14. (canceled)

15. A method of treating a pancreatic tumor in a patient comprising administering to the patient a first agent that is a monoclonal antibody (mAb) that binds and neutralizes human Hepatocyte Growth Factor (HGF) in combination with a second agent that is an inhibitor of platelet-derived growth factor receptor (PDGFR), vascular endothelial growth factor receptor (VEGFR), c-KIT kinase, or FMS-like tyrosine kinase 3 (FLT3).

16. The method of claim 15 wherein the inhibitor is sunitinib.

17. The method of claim 15 wherein the mAb is chimeric, humanized or human and the mAb competes for binding to human HGF with an antibody produced by hybridoma ATCC Number PTA-5162.

18. The method of claim 15 wherein the mAb is a humanized L2G7.

19. The method of claim 15 wherein the first agent is a humanized L2G7 and the second agent is sunitinib.

20-21. (canceled)
